(19) United States
(12) Patent Application Publication
(10) Pub. No.: US 2008/0044365 A1
(43) Pub. Date: Feb. 21, 2008

(54) Concave or annular particles of silicone materials for increasing the sun protection factor of aqueous photoprotective compositions comprising same

(75) Inventors: Jean-Thierry Simonnet, Cachan (FR); Florence L'alloret, Paris (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 11/812,901

(22) Filed: Jun. 22, 2007

Related U.S. Application Data
(60) Provisional application No. 60/819,636, filed on Jul. 11, 2006.

(30) Foreign Application Priority Data
Jun. 22, 2006 (FR) 0652593

(51) Int. Cl. A61K 8/89; A61Q 17/04

(52) U.S. Cl. 424/60; 424/59

(57) ABSTRACT

Sunscreen compositions having increased SPF contain at least one aqueous phase, at least one organic UV screening agent and/or at least one inorganic UV screening agent, notably at least one wetting agent, and an effective SPF-increasing amount of concave or annular particles of a silicone material, formulated into a physiologically acceptable medium therefor.

### CROSS-REFERENCE TO PRIORITY/PROVISIONAL APPLICATIONS

This application claims priority under 35 U.S.C. § 119 of FR 0652593, filed Jun. 22, 2006, and of U.S. Provisional Application No. 60/819,636, filed Jul. 11, 2006, each hereby expressly incorporated by reference in its entirety and each assigned to the assignee hereof.

### BACKGROUND OF THE INVENTION

1. Technical Field of the Invention

The present invention relates to incorporating annular particles of silicone materials, in particular in the form of portions of hollow spheres into compositions comprising, formulated into a physiologically acceptable medium, at least one aqueous phase and at least one organic UV screening agent and/or at least one inorganic UV screening agent for the purpose of increasing the sun protection factor (SPF) of such compositions.

The present invention relates more particularly to compositions comprising, in a physiologically acceptable medium:

a) at least one aqueous phase;

b) concave or annular particles of silicone material, in particular in the form of portions of hollow spheres;

c) at least one wetting agent;

d) at least one organic UV screening agent and/or one inorganic UV screening agent.

2. Description of Background and/or Related and/or Prior Art

It is well known that light radiation with wavelengths of from 280 nm and 400 nm makes possible browning of the human epidermis and that rays with wavelengths of from 280 and 320 nm, known as UV-B radiation, cause erythemas and skin burns which may be harmful to the development of natural tanning; this UV-B radiation must therefore be screened out.

It is also known that UV-A rays, with wavelengths of from 320 and 400 nm, which cause browning of the skin, are capable of bringing about a detrimental change in the latter, in particular in the case of sensitive skin or of skin continually exposed to solar radiation. UV-A rays cause in particular a loss in elasticity of the skin and the appearance of wrinkles, resulting in premature aging. They promote the triggering of the erythemal reaction or accentuate this reaction in certain individuals and can even be the cause of phototoxic or photoallergic reactions. It is therefore desirable also to screen out UV-A radiation.

UV-A and UV-B rays should thus be screened out and there currently exist protective cosmetic compositions for the human epidermis which include UV-A and/or UV-B screening agents in the context of everyday and anti-sun photoprotection.

However, one of the major objectives of these applications is, for cosmetic pleasantness or tolerance reasons but also for formulation reasons, to limit as much as possible the level of sunscreens in the composition.

U.S. Pat. No. 6,039,935 has already provided anti-sun formulations in the form of an oil/water emulsion comprising organic UV screening agents and a non-emulsifying crosslinked elastomeric silicone and volatile silicone for increasing the level of protection and minimizing the amount of screening agent. Oily microgels formed of crosslinked elastomeric silicone are concerned; these microgels have the disadvantages of modifying the viscosity of the composition.

Need exists to find materials which, introduced into the composition, make it possible not only to significantly improve the effectiveness of the sunscreens but without the disadvantages set out above, in particular without increasing the viscosity, and which make it possible to obtain a non-greasy dry feel.

Concave particles of crosslinked organopolysiloxane material which have the form of portions of hollow spheres, obtained by condensation of silanols resulting from the hydrolysis of organosilicone compounds, are known in the prior art, in particular from JP-A-2000-191789, JP-A-2003-128788, EP-1,579,849, EP-1,579,841, FR-2,867,677 and FR-2,877,839. In these patent applications, their use is indicated in particular in cosmetic products for the face or in makeup products, in particular in compact foundation powders or lipstick sticks. In EP-1,642,619 and EP-1,642,620, these same particles have been provided in hair compositions for fixing and/or retaining the form of the hairstyle. Silicone particles of annular shape are also known in U.S. Patent Publication Application No. 2006/0089778, it being possible for these silicone particles to be used as powder in products for caring for the face, makeup products, products for the body, hair products or deodorants.

### SUMMARY OF INVENTION

It has now surprisingly and unexpectedly been determined that the above objective can be achieved by incorporating concave particles of silicone material in an aqueous composition comprising at least one organic UV screening agent and/or one inorganic UV screening agent.

Furthermore, it has now been found that these concave particles of silicone material in combination with a wetting agent in an aqueous composition comprising at least one organic UV screening agent and/or one inorganic UV screening agent also make it possible to significantly increase the photoprotective effectiveness, which is reflected in particular by the sun protection factor (SPF).

The present invention thus features incorporating concave or annular particles of silicone material, in particular in the form of portions of hollow spheres, in an aqueous composition comprising, in a physiologically acceptable medium, at least one aqueous phase and at least one organic UV screening agent and/or one inorganic UV screening agent for the purpose of increasing the sun protection factor (SPF).

The present invention also features incorporating concave or annular particles of silicone material, in particular in the form of portions of hollow spheres, in combination with a wetting agent in an aqueous composition comprising, in a physiologically acceptable medium, at least one organic UV screening agent and/or one inorganic UV screening agent for the purpose of increasing the sun protection factor (SPF).

This invention also features aqueous compositions comprising, in a physiologically acceptable medium:

a) concave or annular particles of silicone material, in particular in the form of portions of hollow spheres;

b) at least one wetting agent;

c) at least one organic UV screening agent and/or one inorganic UV screening agent.

### BRIEF DESCRIPTION OF THE DRAWING

The FIGURE of Drawing illustrates a concave silicone particle in the form of portions of spheres with the shape of a bowl.

### DETAILED DESCRIPTION OF BEST MODE AND SPECIFIC/PREFERRED EMBODIMENTS OF THE INVENTION

The term “physiologically acceptable medium” means a non-toxic medium capable of being applied to the skin, lips, hair, eyelashes, eyebrows or nails. The composition of the invention can constitute in particular a cosmetic or dermatological composition.

The term “wetting agent” means any compound which, introduced at 0.05% (by weight) in aqueous solution, makes it possible to reduce the surface tension of water to a value of less than 35 mN/m and preferably of less than 30 mN/m.

The sun protection factor (SPF) is expressed mathematically by the ratio of the dose of UV radiation necessary to reach the erythemogenic threshold with the UV screening agent to the dose of UV radiation necessary to reach the erythemogenic threshold without UV screening agent.

The term “composition comprising at least one aqueous phase” means a formulation of the oil-in-water (O/W) or water-in-oil (W/O) or multiple (triple: W/O/W or O/W/O) type. The proportion by weight of the aqueous phase preferably varies from 25% to 95%, preferably from 30% to 90% to more preferably still from 40% to 80%. The emulsions of the oil-in-water type or of the water-in-oil-in-water type will more particularly be selected.

Concave or Annular Particles of Silicone Material:

The concave or annular particles present in the compositions according to the invention are silicone particles, in particular particles of portions of hollow spheres composed of a silicone material.

The said particles preferably have a mean diameter of less than or equal to 10 μm, in particular ranging from 0.1 μm to 8 μm, preferably from 0.2 to 7 μm, more preferably ranging from 0.5 to 6 μm and preferably again ranging from 0.5 to 4 μm.

The term “mean diameter” means the greatest dimension of the particle.

Advantageously, these particles have a density of greater than 1.

The portions of hollow spheres used in the compositions according to the invention can have the shape of truncated hollow spheres exhibiting a single orifice communicating with their central cavity and having a transverse cross section with the shape of a horseshoe or arch.

The silicone material is a crosslinked polysiloxane with a three-dimensional structure; it preferably comprises, indeed even is composed of, units of formula (I) SiO₂ and of formula (II) R¹SiO_{1.5} in which R¹ is an organic group having a carbon atom directly connected to the silicon atom.

The organic group R¹ can be a reactive organic group; R¹ can more particularly be an epoxy group, a (meth)acryloyloxy group, an alkenyl group, a mercaptoalkyl, aminoalkyl or haloalkyl group, a glyceroxy group, a ureido group or a cyano group and preferably an epoxy group, a (meth)acryloyloxy group, an alkenyl group or a mercaptoalkyl or aminoalkyl group. These groups generally comprise from 2 to 6 carbon atoms, in particular from 2 to 4 carbon atoms.

The organic group R¹ can also be an unreactive organic group; R¹ can then more particularly be a C₁-C₄ alkyl group, in particular a methyl, ethyl, propyl or butyl group, or a phenyl group and preferably a methyl group.

Exemplary epoxy groups include a 2-glycidoxyethyl group, a 3-glycidoxypropyl group or a 2-(3,4-epoxycyclohexyl)propyl group.

Mention may be made, as (meth)acryloyloxy group, of a 3-methacryloyloxypropyl group or a 3-acryloyloxypropyl group.

Mention may be made, as alkenyl group, of the vinyl, allyl or isopropenyl groups.

Mention may be made, as mercaptoalkyl group, of the mercaptopropyl or mercaptoethyl groups.

Mention may be made, as aminoalkyl group, of a 3-[(2-aminoethyl)amino]propyl group, a 3-aminopropyl group or an N,N-dimethylaminopropyl group.

Mention may be made, as haloalkyl group, of a 3-chloropropyl group or a trifluoropropyl group.

Mention may be made, as glyceroxy group, of a 3-glyceroxypropyl group or a 2-glyceroxyethyl group.

Mention may be made, as ureido group, of a 2-ureidoethyl group.

Mention may be made, as cyano group, of the cyanopropyl or cyanoethyl groups.

Preferably, in the unit of formula (II), R¹ is a methyl group.

Advantageously, the silicone material comprises the units (I) and (II) according to a unit (I)/unit (II) molar ratio ranging from 30/70 to 50/50, preferably ranging from 35/65 to 45/55.

The particles of silicone material can in particular be obtained according to a process comprising:

(a) the introduction into an aqueous medium, in the presence of at least one hydrolysis catalyst and optionally of at least one surfactant, of a compound (III) of formula SiX4 and of a compound (IV) of formula RSiY₃, where X and Y denote, independently of one another, a C₁-C₄ alkoxy group, an alkoxyethoxy group including a C₁-C₄ alkoxy group, a C₂-C₄ acyloxy group, an N,N-dialkylamino group including C₁-C₄ alkyl groups, a hydroxyl group, a halogen atom or a hydrogen atom and R is an organic group comprising a carbon atom connected directly to the silicon atom; and

(b) the operation in which the mixture resulting from stage (a) is brought into contact with an aqueous solution including at least one polymerization catalyst and optionally at least one surfactant, at a temperature of from 30 and 85° C., for at least two hours.

Stage (a) corresponds to a hydrolysis reaction and stage (b) corresponds to a condensation reaction.

In stage (a), the molar ratio of the compound (III) to the compound (IV) generally ranges from 30/70 to 50/50, advantageously from 35/65 to 45/45, and is preferentially 40/60. The ratio by weight of the water to the total of the compounds (III) and (IV) preferably ranges from 10/90 to 70/30. The order of introduction of the compounds (III) and (IV) generally depends on their rate of hydrolysis. The temperature of the hydrolysis reaction generally ranges from 0 to 40° C. and usually does not exceed 30° C. in order to prevent premature condensation of the compounds.

For the X and Y groups of the compounds (III) and (IV):

mention may be made, as C₁-C₄ alkoxy group, of the methoxy or ethoxy groups;

mention may be made, as alkoxyethoxy group including a C₁-C₄ alkoxy group, of the methoxyethoxy or butoxyethoxy groups;

mention may be made, as C₂-C₄ acyloxy group, of the acetoxy or propionyloxy groups;

mention may be made, as N,N-dialkylamino group including a C₁-C₄ alkyl group, of the dimethylamino or diethylamino groups;

mention may be made, as halogen atom, of the chlorine or bromine atoms.

Mention may be made, as compounds of formula (III), of tetramethoxysilane, tetraethoxysilane, tetrabutoxysilane, trimethoxyethoxysilane, tributoxyethoxysilane, tetraacetoxysilane, tetrapropioxysilane, tetra(dimethylamino)silane, tetra(diethylamino)silane, silanetetraol, chlorosilanetriol, dichlorodisilanol, tetrachlorosilane or chlorotrihydrosilane. Preferably, the compound of formula (III) is selected from tetramethoxysilane, tetraethoxysilane, tetrabutoxysilane and their mixtures.

The compound of formula (III) results, after the polymerization reaction, in the formation of the units of formula (I).

The compound of formula (IV) results, after the polymerization reaction, in the formation of the units of formula (II).

The R group in the compound of formula (IV) has the meaning as described for the R¹ group for the compound of formula (II).

Mention may be made, as examples of compounds of formula (IV) comprising an unreactive organic group R, of methyltrimethoxysilane, ethyltriethoxysilane, propyltributoxysilane, butyltributoxysilane, phenyltrimethoxyethoxysilane, methyltributoxyethoxysilane, methyltriacetoxysilane, methyltripropioxysilane, methyltri(dimethylamino)silane, methyltri(diethylamino)silane, methylsilanetriol, methylchlorodisilanol, methyltrichlorosilane or methyltrihydrosilane.

Mention may be made, as examples of compounds of formula (IV) comprising a reactive organic group R, of:

silanes having an epoxy group, such as (3-glycidoxypropyl)trimethoxysilane, (3-glycidoxypropyl)triethoxysilane, [2-(3,4-epoxycyclohexyl)ethyl]trimethoxysilane, (3-glycidoxypropyl)methyldimethoxysilane, (2-glycidoxyethyl)methyldimethoxysilane, (3-glycidoxypropyl)dimethylmethoxysilane or (2-glycidoxyethyl)dimethylmethoxysilane;

silanes having a (meth)acryloyloxy group, such as (3-methacryloyloxypropyl)trimethoxysilane or (3-acryloyloxypropyl)trimethoxysilane;

silanes having an alkenyl group, such as vinyltrimethoxysilane, allyltrimethoxysilane or isopropenyltrimethoxysilane;

silanes having a mercapto group, such as mercaptopropyltrimethoxysilane or mercaptoethyltrimethoxysilane;

silanes having an aminoalkyl group, such as (3-aminopropyl)trimethoxysilane, (3-[(2-aminoethyl)amino]propyl)trimethoxysilane, (N,N-dimethylaminopropyl)trimethoxysilane or (N,N-dimethylaminoethyl)trimethoxysilane;

silanes having a haloalkyl group, such as (3-chloropropyl)trimethoxysilane or trifluoropropyltrimethoxysilane;

silanes having a glyceroxy group, such as (3-glyceroxypropyl)trimethoxysilane or di(3-glyceroxypropyl)dimethoxysilane;

silanes having a ureido group, such as (3-ureidopropyl)trimethoxysilane, (3-ureidopropyl)methyldimethoxysilane or (3-ureidopropyl)dimethylmethoxysilane;

silanes having a cyano group, such as cyanopropyltrimethoxysilane, cyanopropylmethyldimethoxysilane or cyanopropyldimethylmethoxysilane.

Preferably, the compound of formula (IV) comprising a reactive organic group R is selected from silanes having an epoxy group, silanes having a (meth)acryloyloxy group, silanes having an alkenyl group, silanes having a mercapto group or silanes having an aminoalkyl group.

Examples of compounds (III) and (IV) which are preferred for the implementation of this invention are respectively tetraethoxysilane and methyltrimethoxysilane.

Use may independently be made, as hydrolysis and polymerization catalysts, of basic catalysts, such as sodium hydroxide, potassium hydroxide, sodium carbonate, sodium hydrogencarbonate, ammonia or amines, such as trimethylamine, triethylamine or tetramethylammonium hydroxide, or acidic catalysts, such as organic acids, for example citric acid, acetic acid, methanesulfonic acid, p-toluenesulfonic acid, dodecylbenzenesulfonic acid or dodecylsulfonic acid, or inorganic acids, such as hydrochloric acid, sulfuric acid or phosphoric acid.

When it is present, the surfactant used is preferably a nonionic or anionic surfactant or a mixture of the two. Sodium dodecylbenzenesulfonate can be used as anionic surfactant. The end of the hydrolysis is marked by the disappearance of the products (III) and (IV), which are insoluble in water, and the production of a homogeneous liquid layer.

The condensation stage (b) can use the same catalyst as the hydrolysis stage or another catalyst selected from those mentioned above.

On conclusion of this process, a suspension in water of fine organosilicone particles is obtained, which particles can optionally be separated subsequently from their medium. The process described above can thus comprise an additional stage of filtration, for example on a membrane filter, of the product resulting from stage (b), optionally followed by a stage of centrifuging the filtrate, intended to separate the particles from the liquid medium, and then by a stage of drying the particles. Other separation methods can, of course, be employed.

The shape of the portions of hollow spheres obtained according to the above process and their dimensions will depend in particular on the method used to bring the products into contact in stage (b).

A somewhat basic pH and introduction under cold conditions of the polymerization catalyst into the mixture resulting from stage (a) will result in portions of hollow spheres with the shape of round-bottomed “bowls”, whereas a somewhat acidic pH and dropwise introduction of the mixture resulting from stage (a) into the hot polymerization catalyst will result in portions of hollow spheres having a transverse cross section with the shape of a “horseshoe”.

According to a preferred embodiment of the invention, portions of hollow spheres with the shape of “bowls” are used. These can be obtained as described in JP-A-2003-128788.

Portions of hollow spheres with the shape of a horseshoe are described in JP-A-2000-191789.

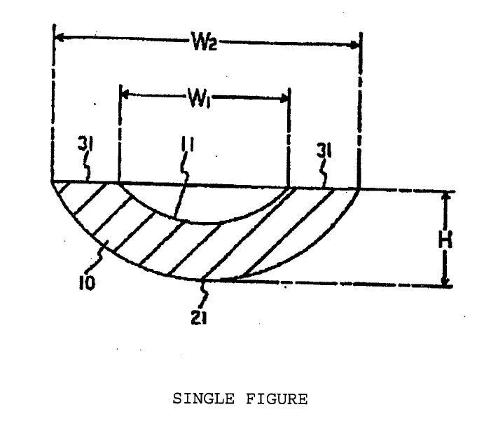

A concave particle in the form of portions of spheres with the shape of a bowl is illustrated in transverse cross section in the appended FIG. 1. The width W2 corresponds to the diameter of the particles.

As emerges from this FIGURE, these concave particles are formed (in cross section perpendicular to the plane of the opening delimited by the portion of hollow sphere) of a small internal arc (11), of a large external arc (21) and of segments (31) which connect the ends of the respective arcs, the width (W1) from the two ends of the small internal arc (11) ranging from 0.01 to 8 μm, preferably from 0.02 to 6 μm, on average, the width (W2) from the two ends of the large external arc (21) ranging from 0.05 to 10 μm, preferably from 0.06 to 8 μm, on average, and the height (H) of the large external arc (21) ranging from 0.015 to 8 μm, preferably from 0.03 to 6 μm, on average.

The dimensions indicated above are obtained by calculating the mean of the dimensions of one hundred particles selected on an image obtained with a scanning electron microscope.

Mention may be made, as concave particles in the form of portions of spheres which can be used according to the invention, of, for example:

particles composed of the crosslinked organosilicone TAK-110 (crosslinked methylsilanol/silicate polymer) from Takemoto Oil & Fat, with the shape of a bowl, with a width of 2.5 μm, a height of 1.2 μm and a thickness of 150 nm (particles marketed under the trademark NLK-506 by Takemoto Oil & Fat);

particles composed of the crosslinked organosilicone TAK-110 (crosslinked methylsilanol/silicate polymer) from Takemoto Oil & Fat, with the shape of a bowl, with a width of 0.8 μm, a height of 0.4 μm and a thickness of 130 nm (particles marketed under the trademark NLK-515 by Takemoto Oil & Fat);

particles composed of the crosslinked organosilicone TAK-110 (crosslinked methylsilanol/silicate polymer) from Takemoto Oil & Fat, with the shape of a bowl, with a width of 7 μm, a height of 3.5 μm and a thickness of 200 nm (particles marketed under the trademark NLK-510 by Takemoto Oil & Fat).

These particles have the INCI name: methylsilanol/silicate crosspolymer.

Advantageously, the concave silicone particles have a mean diameter of less than or equal to 5 μm, in particular ranging from 0.1 μm to 5 μm, preferably ranging from 0.2 to 5 μm, more preferably ranging from 0.5 to 4 μm and preferably again ranging from 0.5 to 3 μm.

These particles make possible, in addition to the reduction, indeed even the elimination, of the sticky feel, the optimization of the properties of slip, of spreading and of comfort of the composition according to the invention.

The silicone particles of annular shape are preferably selected from those described and synthesized in U.S. Patent Publication Application No. 2006/0089478. They exhibit a mean external diameter of 0.05 to 15 μm and a mean internal diameter of 0.01 to 10 μm, the difference from the mean external diameter and the mean internal diameter being from 0.04 to 5 μm.

They exhibit a polysiloxane network comprising siloxane units of formulae (1), (2), (3), (4), (5) and (6):
SiO_{4/2} (1)
Si(OH)_{3/2} (2)
R₁SiO_{3/2} (3)
R₂SiO_{3/2} (4)
R₃SiO_{3/2} (5)
R₄SiO_{3/2} (6)
in which:

R¹, and R₃ denote unreactive hydrocarbon groups, in particular alkyl, cycloalkyl, aryl, alkylaryl or aralkyl groups, preferably C₁-C₃ alkyl groups, in particular methyl, ethyl or propyl groups and preferably a methyl group,

and R₂ and R₄ each denote a hydrocarbon group selected from the acryloyloxy, methacryloyloxy, vinyl or mercapto groups;

the siloxane units of formula (1)/siloxane units of formulae (2), (3), (4), (5) and (6) molar ratio being from 20/80 to 50/50;

the siloxane units of formulae (2), (3) and (4)/siloxane units of formulae (5) and (6) molar ratio being from 50/50 to 75/25;

the siloxane units of formulae (3) and (5)/siloxane units of formulae (4) and (6) molar ratio being from 20/80 to 60/40.

Mention may be made, as acryloyloxy group, of a 2-methacryloyloxyethyl group or a 3-acryloyloxypropyl group.

Mention may be made, as (meth)acryloyloxy group, of a 3-methacryloyloxypropyl group or a 3-acryloyloxypropyl group.

Mention may be made, as mercaptoalkyl group, of a mercaptopropyl or mercaptoethyl group.

Mention may be made, as vinyl group, of the allyl, isopropenyl or 2-methylallyl groups.

The concave or annular silicone particles can be present in the compositions according to the invention in a content ranging from 0.1 to 15% by weight, preferably ranging from 0.5 to 10% by weight and preferably ranging from 0.5 to 7.5% by weight, with respect to the total weight of the composition.

UV Screening Agents:

The screening compositions in accordance with the invention comprise organic and/or inorganic UV screening agents active in the UV-A and/or UV-B region which are hydrophilic and/or lipophilic and/or properly insoluble in the cosmetic solvents commonly used.

The hydrophilic, lipophilic or insoluble organic UV screening agents are selected in particular from anthranilates; dibenzoylmethane derivatives; cinnamic derivatives; salicylic derivatives; camphor derivatives; benzophenone derivatives; β,β-diphenylacrylate derivatives; triazine derivatives; benzotriazole derivatives; benzalmalonate derivatives, in particular those cited in U.S. Pat. No. 5,624,663; benzimidazole derivatives; imidazolines; bis-benzoazolyl derivatives, such as described in EP-669,323 and U.S. Pat. No. 2,463,264; p-aminobenzoic acid (PABA) derivatives; methylenebis(hydroxyphenylbenzotriazole) derivatives, such as described in U.S. Pat. Nos. 5,237,071, 5,166,355, GB-2,303,549, DE-197,26,184 and EP-893,119; benzoxazole derivatives, such as described in EP-0,832,642, EP-1,027,883, EP-1,300,137 and DE-10162844; screening polymers and screening silicones, such as those described in particular in WO 93/04665; dimers derived from α-alkylstyrene, such as those described in DE-19855649; 4,4-diarylbutadienes, such as described in EP-0,967,200, DE-19746654, DE-19755649, EP-A-1,008,586, EP-1,133,980 and EP-133,981; and their mixtures.

Mention may be made, as examples of organic UV screening agents, of those denoted below under their INCI names:

para-Aminobenzoic acid derivatives:

PABA,

Ethyl PABA,

Ethyl Dihydroxypropyl PABA,

Ethylhexyl Dimethyl PABA, marketed in particular under the trademark “Escalol 507” by ISP,

Glyceryl PABA,

PEG-25 PABA, marketed under the trademark “Uvinul P25” by BASF,

Dibenzoylmethane Derivatives:

Butyl Methoxydibenzoylmethane, marketed in particular under the trademark “Parsol 1789” by Hoffmann-LaRoche,

Isopropyl Dibenzoylmethane,

Salicylic Derivatives:

Homosalate, marketed under the trademark “Eusolex HMS” by Rona/EM Industries,

Ethylhexyl Salicylate, marketed under the trademark “Neo Heliopan OS” by Haarmann and Reimer,

Dipropyleneglycol Salicylate, marketed under the trademark “Dipsal” by Scher,

TEA Salicylate, marketed under the trademark “Neo Heliopan TS” by Haarmann and Reimer,

Cinnamic Derivatives:

Ethylhexyl Methoxycinnamate, marketed in particular under the trademark “Parsol MCX” by Hoffmann-LaRoche,

Isopropyl Methoxycinnamate,

Isoamyl Methoxycinnamate, marketed under the trademark “Neo Heliopan E 1000” by Haarmann and Reimer,

Cinoxate,

DEA Methoxycinnamate,

Diisopropyl Methylcinnamate,

Glyceryl Ethylhexanoate Dimethoxycinnamate,

β,β-Diphenylacrylate Derivatives:

Octocrylene, marketed in particular under the trademark “Uvinul N539” by BASF,

Etocrylene, marketed in particular under the trademark “Uvinul N35” by BASF,

Benzophenone Derivatives:

Benzophenone-1, marketed under the trademark “Uvinul 400” by BASF,

Benzophenone-2, marketed under the trademark “Uvinul D50” by BASF,

Benzophenone-3 or Oxybenzone, marketed under the trademark “Uvinul M40” by BASF,

Benzophenone-4, marketed under the trademark “Uvinul MS40” by BASF,

Benzophenone-5,

Benzophenone-6, marketed under the trademark “Helisorb 11” by Norquay,

Benzophenone-8, marketed under the trademark “Spectra-Sorb UV-24” by American Cyanamid,

Benzophenone-9, marketed under the trademark “Uvinul DS-49” by BASF, Benzophenone-12,

n-Hexyl 2-(4-diethylamino-2-hydroxybenzoyl)benzoate,

Benzylidenecamphor Derivatives:

3-Benzylidene camphor, manufactured under the trademark “Mexoryl SD” by Chimex,

4-Methylbenzylidene camphor, marketed under the trademark “Eusolex 6300” by Merck,

Benzylidene Camphor Sulfonic Acid, manufactured under the trademark “Mexoryl SL” by Chimex,

Camphor Benzalkonium Methosulfate, manufactured under the trademark “Mexoryl SO” by Chimex,

Terephthalylidene Dicamphor Sulfonic Acid, manufactured under the trademark “Mexoryl SX” by Chimex,

Polyacrylamidomethyl Benzylidene Camphor, manufactured under the trademark “Mexoryl SW” by Chimex,

Phenylbenzimidazole Derivatives:

Phenylbenzimidazole Sulfonic Acid, marketed in particular under the trademark “Eusolex 232” by Merck,

Disodium Phenyl Dibenzimidazole Tetrasulfonate, marketed under the trademark “Neo Heliopan AP” by Haarmann and Reimer,

Phenylbenzotriazole Derivatives:

Drometrizole Trisiloxane, marketed under the trademark “Silatrizole” by Rhodia Chimie,

Methylene Bis-Benzotriazolyl Tetramethylbutylphenol, marketed in the solid form under the trademark “Mixxim BB/100” by Fairmount Chemical or in the micronized form in aqueous dispersion under the trademark “Tinosorb M” by Ciba Specialty Chemicals,

Triazine Derivatives:

Bis-Ethylhexyloxyphenol Methoxyphenyl Triazine, marketed under the trademark “Tinosorb S” by Ciba-Geigy,

Ethylhexyl Triazone, marketed in particular under the trademark “Uvinul T150” by BASF,

Diethylhexyl Butamido Triazone, marketed under the trademark “Uvasorb HEB” by Sigma 3V,

2,4,6-Tris(dineopentyl 4′-aminobenzalmalonate)-s-triazine, the symmetrical triazine screening agents described in U.S. Pat. No. 6,225,467, WO 2004/085412 (see compounds 6 and 9) or the document “Symmetrical Triazine Derivatives”, IP.COM Journal, IP.COM INC, WEST HENRIETTA, NY, US (20 Sep. 2004), in particular the 2,4,6-tris(biphenyl)-1,3,5-triazines (especially 2,4,6-tris(biphenyl-4-yl)-1,3,5-triazine) and 2,4,6-tris(terphenyl)-1,3,5-triazine, which is taken up again in WO 06/035000, WO 06/034982, WO 06/034991, WO 06/035007, WO 2006/034992 and WO 2006/034985.

Anthranilic Derivatives:

Menthyl anthranilate, marketed under the trademark “Neo Heliopan MA” by Haarmann and Reimer,

Imidazoline Derivatives:

Ethylhexyl Dimethoxybenzylidene Dioxoimidazoline Propionate,

Benzalmalonate Derivatives:

Dineopentyl 4′-methoxybenzalmalonate,

Polyorganosiloxane comprising benzalmalonate functional groups, such as Polysilicone-15, marketed under the trademark “Parsol SLX” by Hoffmann-LaRoche,

4,4-Diarylbutadiene Derivatives:

1,1-Dicarboxy(2,2′-dimethylpropyl)-4,4-diphenylbutadiene,

Benzoxazole Derivatives:

2,4-bis[5-1 (dimethylpropyl)benzoxazol-2-yl-(4-phenyl)imino]-6-(2-ethylhexyl)imino-1,3,5-triazine, marketed under the trademark of Uvasorb K2A by Sigma 3V

and their mixtures.

The preferred organic UV screening agents are selected from:

Ethylhexyl Methoxycinnamate,

Homosalate,

Ethylhexyl Salicylate,

Octocrylene,

Phenylbenzimidazole Sulfonic Acid,

Benzophenone-3,

Benzophenone-4,

Benzophenone-5,

n-Hexyl 2-(4-diethylamino-2-hydroxybenzoyl)benzoate,

4-Methylbenzylidene Camphor,

Terephthalylidene Dicamphor Sulfonic Acid,

Disodium Phenyl Dibenzimidazole Tetrasulfonate,

Ethylhexyl triazone,

Bis-Ethylhexyloxyphenol Methoxyphenyl Triazine,

Diethylhexyl Butamido Triazone,

2,4,6-Tris(dineopentyl 4′-aminobenzalmalonate)-s-triazine,

2,4,6-Tris(diisobutyl 4′-aminobenzalmalonate)-s-triazine,

2,4,6-Tris(biphenyl-4-yl)-1,3,5-triazine,

2,4,6-Tris(terphenyl)-1,3,5-triazine,

Methylene Bis-Benzotriazolyl Tetramethylbutylphenol,

Drometrizole Trisiloxane,

Polysilicone-15,

Dineopentyl 4′-methoxybenzalmalonate,

1,1-Dicarboxy(2,2′-dimethylpropyl)-4,4-diphenylbutadiene,

2,4-Bis[5-1 (dimethylpropyl)benzoxazol-2-yl-(4-phenyl)imino]-6-(2-ethylhexyl)imino-1,3,5-triazine,

and their mixtures.

The inorganic screening agents are selected from pigments (mean size of the primary particles: generally from 5 nm and 100 nm, preferably from 10 nm and 50 nm) formed of metal oxides which may or may not be coated, such as, for example, pigments formed of titanium oxide (amorphous or crystalline in the rutile and/or anatase form), iron oxide, zinc oxide, zirconium oxide or cerium oxide, which are all UV photoprotective agents well known per se.

The pigments may or may not be coated.

The coated pigments are pigments which have been subjected to one or more surface treatments of chemical, electronic, mechanochemical and/or mechanical nature with compounds such as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminum salts of fatty acids, metal alkoxides (titanium or aluminum alkoxides), polyethylene, silicones, proteins (collagen, elastin), alkanolamines, silicon oxides, metal oxides or sodium hexametaphosphate.

In known manner, the silicones are organosilicon polymers or oligomers comprising a linear or cyclic and branched or crosslinked structure, of variable molecular weight, obtained by polymerization and/or polycondensation of suitable functional silanes and essentially composed of a repetition of main units in which the silicon atoms are connected to one another via oxygen atoms (siloxane bond), optionally substituted hydrocarbon radicals being connected directly to the said silicon atoms via a carbon atom.

The term “silicones” also encompasses the silanes necessary for their preparation, in particular alkylsilanes.

The silicones used for the coating of the pigments suitable for the present invention are preferably selected from the group consisting of alkylsilanes, polydialkylsiloxanes and polyalkylhydrosiloxanes. More preferably still, the silicones are selected from the group consisting of octyltrimethylsilane, polydimethylsiloxanes and polymethylhydrosiloxanes.

Of course, the pigments formed of metal oxides may, before their treatment with silicones, have been treated with other surfacing agents, in particular with cerium oxide, alumina, silica, aluminum compounds, silicon compounds or their mixtures.

The coated pigments are more particularly titanium oxides coated:

with silica, such as the product “Sunveil” from Ikeda,

with silica and with iron oxide, such as the product “Sunveil F” from Ikeda,

with silica and with alumina, such as the products “Microtitanium Dioxide MT 500 SA” and “Microtitanium Dioxide MT 100 SA” from Tayca, “Tioveil” from Tioxide and “Mirasun TiW 60” from Rhodia,

with alumina, such as the products “Tipaque TTO-55 (B)” and “Tipaque TTO-55 (A)” from Ishihara and “UVT 14/4” from Kemira,

with alumina and with aluminum stearate, such as the product “Microtitanium Dioxide MT 100 T, MT 100 TX, MT 100 Z or MT-01” from Tayca, the products “Solaveil CT-10 W” and “Solaveil CT 100” from Uniqema and the product “Eusolex T-AVO” from Merck,

with silica, with alumina and with alginic acid, such as the product “MT-100 AQ” from Tayca,

with alumina and with aluminum laurate, such as the product “Microtitanium Dioxide MT 100 S” from Tayca,

with iron oxide and with iron stearate, such as the product “Microtitanium Dioxide MT 100 F” from Tayca,

with zinc oxide and with zinc stearate, such as the product “BR351” from Tayca,

with silica and with alumina and treated with a silicone, such as the products “Microtitanium Dioxide MT 600 SAS”, “Microtitanium Dioxide MT 500 SAS” or “Microtitanium Dioxide MT 100 SAS” from Tayca,

with silica, with alumina and with aluminum stearate and treated with a silicone, such as the product “STT-30-DS” from Titan Kogyo,

with silica and treated with a silicone, such as the product “UV-Titan X 195” from Kemira,

with alumina and treated with a silicone, such as the products “Tipaque TTO-55 (S)” from Ishihara or “UV Titan M 262” from Kemira,

with triethanolamine, such as the product “STT-65-S” from Titan Kogyo,

with stearic acid, such as the product “Tipaque TTO-55 (C)” from Ishihara,

with sodium hexametaphosphate, such as the product “Microtitanium Dioxide MT 150 W” from Tayca.

Other titanium oxide pigments treated with a silicone are preferably TiO₂ treated with octyltrimethylsilane and for which the mean size of the individual particles is from 25 and 40 nm, such as that marketed under the trademark “T 805” by Degussa Silices, TiO₂ treated with a polydimethylsiloxane and for which the mean size of the individual particles is 21 nm, such as that marketed under the trademark “70250 Cardre UF TiO2SI3” by Cardre, anatase/rutile TiO₂ treated with a polydimethylhydrosiloxane and for which the mean size of the individual particles is 25 nm, such as that marketed under the trademark “Microtitanium Dioxide USP Grade Hydrophobic” by Color Techniques.

The uncoated titanium oxide pigments are, for example, marketed by Tayca under the trademarks “Microtitanium Dioxide MT 500 B” or “Microtitanium Dioxide MT600 B”, by Degussa under the trademark “P 25”, by Wacker under the trademark “Oxyde de titane transparent PW”, by Miyoshi Kasei under the trademark “UFTR”, by Tomen under the trademark “ITS” and by Tioxide under the trademark “Tioveil AQ”.

The uncoated zinc oxide pigments are, for example:

those marketed under the trademark “Z-cote” by Sunsmart;

those marketed under the trademark “Nanox” by Elementis;

those marketed under the trademark “Nanogard WCD 2025” by Nanophase Technologies.

The coated zinc oxide pigments are, for example:

those marketed under the trademark “Oxide zinc CS-5” by Toshibi (ZnO coated with polymethylhydrosiloxane);

those marketed under the trademark “Nanogard Zinc Oxide FN” by Nanophase Technologies (as a 40% dispersion in Finsolv TN, C₁₂-C₁₅ alkyl benzoate);

those marketed under the trademark “Daitopersion Zn-30” and “Daitopersion Zn-50” by Daito (dispersions in oxyethylenated polydimethylsiloxane/cyclopolymethylsiloxane comprising 30% or 50% of zinc nanooxides coated with silica and polymethylhydrosiloxane);

those marketed under the trademark “NFD Ultrafine ZnO” by Daikin (ZnO coated with phosphate of perfluoroalkyl and copolymer based on perfluoroalkylethyl as a dispersion in cyclopentasiloxane);

those marketed under the trademark “SPD-Z1” by Shin-Etsu (ZnO coated with silicone-grafted acrylic polymer dispersed in cyclodimethylsiloxane);

those marketed under the trademark “Escalol Z100” by ISP (alumina-treated ZnO dispersed in the ethylhexyl methoxycinnamate/PVP-hexadecene copolymer/methicone mixture);

those marketed under the trademark “Fuji ZnO-SMS-10” by Fuji Pigment (ZnO coated with silica and polymethylsilsesquioxane); those marketed under the trademark “Nanox Gel TN” by Elementis (ZnO dispersed at 55% in C₁₂-C₁₅ alkyl benzoate with hydroxystearic acid polycondensate).

The uncoated cerium oxide pigments are marketed, for example, under the trademark “Colloidal Cerium Oxide” by Rhone-Poulenc.

The uncoated iron oxide pigments are, for example, marketed by Arnaud under the trademarks “Nanogard WCD 2002 (FE 45B)”, “Nanogard Iron FE 45 BL AQ”, “Nanogard FE 45R AQ” or “Nanogard WCD 2006 (FE 45R)”, or by Mitsubishi under the trademark “TY-220”.

The coated iron oxide pigments are, for example, marketed by Arnaud under the trademarks “Nanogard WCD 2008 (FE 45B FN)”, “Nanogard WCD 2009 (FE 45B 556)”, “Nanogard FE 45 BL 345” or “Nanogard FE 45 BL” or by BASF under the trademark “Oxyde de fer transparent”.

Mention may also be made of mixtures of metal oxides, in particular of titanium dioxide and of cerium dioxide, including the mixture of equal weights of titanium dioxide coated with silica and of cerium dioxide coated with silica marketed by Ikeda under the trademark “Sunveil A”, and also the mixture of titanium dioxide and of zinc dioxide coated with alumina, with silica and with silicone, such as the product “M 261” marketed by Kemira, or coated with alumina, with silica and with glycerol, such as the product “M 211” marketed by Kemira.

The UV screening agents are generally present in the compositions according to the invention in proportions ranging from 0.01% to 20% by weight, with respect to the total weight of the composition, and preferably ranging from 0.1% to 10% by weight, with respect to the total weight of the composition.

According to a particularly preferred embodiment of the invention, the compositions additionally comprise at least one wetting agent.

The wetting agents in accordance with the invention are preferably selected from water-soluble silicones comprising at least one terminal or pendent monovalent polyoxyalkylene group and which, introduced at 0.05% by weight into an aqueous solution, are capable of reducing the surface tension of water to a value of less than 35 mN/m and preferably of less than 30 mN/m.

The wetting agents in accordance with the invention are more preferably selected from water-soluble silicones comprising at least one polyoxyalkylene group of following general formula (a):
R²₃SiO(R²₂SiO)ₚ(R²PESiO)_{q}SiR²₃ (a)
in which:

the R² radicals, which are identical or different, denote a monovalent hydrocarbon radical selected from alkyl, aryl and aralkyl radicals having at most 10 carbon atoms; some of the R² radicals can also additionally comprise an ethylcyclohexylene monoxide group of formula: 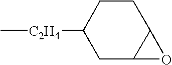
and are in low proportion in the polysiloxane chain;

p varies from 0 to 150, preferably from 0 to 100 and more preferably from 0 to 30;

q varies from 1 to 12, preferably from 1 to 10 and more preferably from 1 to 8;

the polyether group PE has the following formula (b):
—CₓH₂ₓ(OC₂H₄)_{y}(OC₃H₆)_{z}OR³ (b)
in which:

x varies from 1 to 8 and preferably varies from 2 to 4 and more preferably is equal to 3;

y is greater than 0;

z is greater than or equal to 0; the values of y and z are such that the total molecular weight of the polyoxyalkylene portion of the polyether group PE varies from 200 to 10,000 and more preferably from 350 to 4,000;

R³ is hydrogen, a C₁-C₈ alkyl group or a C₂-C₈ acyl group.

It should be noted that, when z is other than 0, the polyoxyethylene and polyoxypropylene units can be distributed randomly along the polyether chain PE or distributed as blocks or indeed simultaneously distributed as blocks and randomly.

Preferably, the R² radicals are selected from lower C₁-C₆ alkyls, such as methyl, ethyl, butyl or hexyl, phenyl and benzyl. More particularly, the R² radicals are selected from lower C₁-C₄ alkyls and more particularly still denote methyl.

Preferably, the R³ radicals are selected from lower C₁-C₄ alkyls and more particularly still denote methyl.

The number of oxyethylene units of the PE group must be sufficient to produce a cloud point in water from 25 and 90° C. and more preferably from 40 to 70° C.

The water-soluble silicones of formula (a) can be obtained according to the process described in U.S. Pat. No. 4,847,398.

Use will preferably be made, among the water-soluble silicones of formula (a), of that of following formula (a′):
Me₃SiO(MeSiO)ₚ(MePESiO)_{q}SiMe₃ (a′)
where Me is methyl; PE is:
—(CH₂)₃(OC₂H₄)_{y}(OC₃H₆)_{z}OR³ (b′)
where x, z and z have the same values indicated above and R³ is hydrogen or a C₁-C₄ alkyl group and more particularly methyl.

Mention may be made, as other family of water-soluble silicones which can be used according to the invention, of the branched silicones of following formula (c):
(MeSiO)_{q-2}[SiOMe₂)_{p/q}OPE]_{q} (c)
where p and q have the same values indicated above in the formula (a); Me signifies methyl; PE is the group of following formula (d):
—(OC₂H₄)_{y}(OC₃H₆)_{z}R³ (d)
where y and z have the same values indicated above in the formula (b) and R³ is a C₁-C₄ alkyl group and more particularly methyl.

Such silicones are, for example, marketed by OSI under the trademarks Silwet L-720®, Silwet L-7002®, Silwet L-7600®, Silwet L-7604®, Silwet L-7605®, Silwet L-7607®, Silwet 1614, Silwet L-7657®, Silwet L-7200®, Silwet L7230, Silsoft 305, Silsoft 820 and Silsoft 880 or by Goldschmidt under the trademarks Tegowet 260®, Tegowet 500®, Tegowet-505® and Tegowet 510®.

Values of surface tensions at 25° C. of aqueous solutions comprising 0.05% (by weight) of different wetting agents are collated in the following table.

| | Surface tension at 0.05% |
|---|---|
| Wetting agent | in water (mN/m), 25° C. |
| Tegowet 500 | 33 |
| Tegowet 510 | 29 |
| Silsoft 880 | 26 |
| Silsoft 305 | 21 |

According to the invention, the wetting agent or agents are present at concentrations ranging from 0.01% to 10% by weight, preferably from 0.05% to 5% by weight and more particularly from 0.1% to 3% by weight, with respect to the total weight of the final composition.

The compositions according to the invention can be prepared according to the techniques for the preparation of emulsions well known to a person skilled in the art. They are generally provided in the form of a simple or complex emulsion (O/W, W/O, O/W/O or W/O/W).

The compositions which can be used according to the invention can be more or less fluid and have the appearance of a white or colored cream, of an ointment, of a milk, of a lotion, of a serum or of a paste. They can also be provided in the solid form, for example in the stick form. They can optionally be packaged in an aerosol and be provided in the foam or spray form.

The choice will more particularly be made of the emulsions of the oil-in-water type or of the water-in-oil-in-water type.

The compositions in accordance with the invention generally comprise at least one oily phase which comprises at least one oil, in particular a cosmetic oil. The term “oil” is understood to mean a fatty substance which is liquid at ambient temperature (25° C.).

Use may be made, as oils which can be used in the composition of the invention, for example, of hydrocarbon oils of animal origin, such as perhydrosqualene (or squalane); hydrocarbon oils of vegetable origin, such as triglycerides of caprylic/capric acids, for example those marketed by Stearineries Dubois or those marketed under the trademarks Miglyol 810, 812 and 818 by Dynamit Nobel, or oils of vegetable origin, for example sunflower, maize, soybean, cucumber, grape seed, sesame, hazelnut, apricot, macadamia, arara, coriander, castor, avocado or jojoba oil or shea butter oil; synthetic oils; silicone oils, such as volatile or non-volatile polymethylsiloxanes (PDMSs) comprising a linear or cyclic silicone chain which are liquid or pasty at ambient temperature; fluorinated oils, such as those which are partially hydrocarbon and/or silicone, for example those described in JP-A-2-295912; ethers, such as dicaprylyl ether (CTFA name); and esters, such as benzoate C₁₂-C₁₅ fatty alcohols (Finsolv TN from Finetex); arylalkyl benzoate derivatives, such as 2-phenylethyl benzoate (X-Tend 226 from ISP); amidated oils, such as isopropyl N-lauroylsarcosinate (Eldew SL-205 from Ajinomoto), and their mixtures.

The oily phase can also comprise one or more fatty substances selected, for example, from fatty alcohols (cetyl alcohol, stearyl alcohol, cetearyl alcohol), fatty acids (stearic acid) or waxes (paraffin wax, polyethylene waxes, carnauba wax, beeswax). The oily phase can comprise lipophilic gelling agents, surfactants or also organic or inorganic particles. The oily phase can preferably represent from 1 to 70% of oil by weight, with respect to the total weight of the composition.

The compositions according to the invention in the emulsion form generally comprise emulsifying surfactants which are appropriately selected according to the type of emulsion selected. When the emulsion is a triple emulsion, it generally comprises an emulsifier in the primary emulsion and an emulsifier in the external phase into which the primary emulsion is introduced.

In the case of the simple oil-in-water emulsion, the dispersed oily phase can represent from 1% to 70% by weight, preferably from 5% to 60% by weight and better still from 10% to 50% by weight, with respect to the total weight of the simple emulsion.

In the case of the water-in-oil-in-water triple emulsion, the W/O primary emulsion can represent, for example, from 5% to 70% by weight, preferably from 10% to 60% by weight and better still from 15% to 50% by weight, with respect to the total weight of the triple emulsion. The internal aqueous phase of the W/O primary emulsion preferably is from 5% to 90%, better still from 30% to 90% to even better still from 40% to 80% of the total weight of the primary emulsion.

The emulsifying surfactants are generally present at a minimum concentration of 0.5% by weight, with respect to the total weight of the composition.

They are preferably used at proportions ranging from 0.5% to 30% by weight, preferably from 0.5% to 20% by weight and better still from 0.5% to 15% by weight, with respect to the total weight of the composition.

Mention may be made, as emulsifying surfactants which can be used for the preparation of the W/O emulsions, for example, of sorbitan, glycerol or sugar alkyl esters or ethers; silicone surfactants, such as dimethicone copolyols, for example the mixture of cyclomethicone and of dimethicone copolyol marketed under the trademark “DC 5225 C” by Dow Corning, and alkyl dimethicone copolyols, such as lauryl methicone copolyol, marketed under the trademark “Dow Corning 5200 Formulation Aid” by Dow Corning, cetyl dimethicone copolyol, such as the product marketed under the trademark Abil EM 90R by Goldschmidt, and the mixture of cetyl dimethicone copolyol, of polyglycerol (4 mol) isostearate and of hexyl laurate marketed under the trademark Abil WE O9 by Goldschmidt. It is also possible to add thereto one or more coemulsifiers which, advantageously, can be selected from the group consisting of polyol alkyl esters.

Mention may in particular be made, as polyol alkyl esters, of polyethylene glycol esters, such as PEG-30 dipolyhydroxystearate, such as the product marketed under the trademark Arlacel P135 by ICI.

Mention may be made, as esters of glycerol and/or of sorbitan, for example, of polyglycerol isostearate, such as the product marketed under the trademark Isolan GI 34 by Goldschmidt; sorbitan isostearate, such as the product marketed under the trademark Arlacel 987 by ICI; glycerol sorbitan isostearate, such as the product marketed under the trademark Arlacel 986 by ICI, and their mixtures.

Mention may be made, for the O/W emulsions, for example, as emulsifiers, of nonionic emulsifiers, such as oxyalkylenated (more particularly polyoxyethylenated) esters of fatty acids and of glycerol; oxyalkylenated esters of fatty acids and of sorbitan; oxyalkylenated (oxyethylenated and/or oxypropylenated) fatty acid esters, such as the PEG-100 stearate/glyceryl stearate mixture marketed, for example, by ICI under the trademark Arlacel 165; oxyalkylenated (oxyethylenated and/or oxypropylenated) fatty alcohol ethers; sugar esters, such as sucrose stearate; ethers of fatty alcohol and of sugar, in particular alkyl polyglucosides (APG), such as decyl glucoside and lauryl glucoside, for example marketed by Henkel under the respective names Plantaren 2000 and Plantaren 1200, cetearyl glucoside, optionally as a mixture with cetearyl alcohol, for example marketed under the trademark Montanov 68 by Seppic, under the trademark Tegocare CG90 by Goldschmidt and under the trademark Emulgade KE3302 by Henkel, and arachidyl glucoside, for example in the form of the mixture of arachidyl and behenyl alcohols and of arachidyl glucoside marketed under the trademark Montanov 202 by Seppic. According to a specific embodiment of the invention, the mixture of the alkyl polyglucoside as defined above with the corresponding fatty alcohol can be in the form of a self-emulsifying composition, for example as disclosed in WO-A-92/06778.

When an emulsion is involved, the aqueous phase of the latter can comprise a nonionic vesicular dispersion prepared according to known methods (Bangham, Standish and Watkins, J. Mol. Biol., 13, 238 (1965), FR-2,315,991 and FR-2,416,008).

According to a specific embodiment of the invention, the emulsions according to the invention can comprise only 1% by weight or less and can even be devoid of emulsifying surfactants while being stable on storage. In this case, they can be stabilized by various techniques, such as the use of hydrophilic or lipophilic thickening agents, such as those of EP-864,320, of amphiphilic polymers, such as those cited in EP-1,093,796 or in WO 02/44231, or of solid particles (Pickering-type emulsions), such as the emulsions cited in WO 98/42300, WO 98/42301, EP-987,001, EP-987,002, EP-987,003, EP-987,004, EP-987,005, EP-987,006, EP-987,007, EP-987,008, WO 2000/07548, WO 2000/07549 or EP-992,233.

In the W/O/W triple emulsions according to the invention, the W/O primary emulsion advantageously comprises at least one emulsifier with an HLB of less than 10 (HLP=hydrophilic-lipophilic balance). The emulsifiers can be selected, for example, from the group consisting of alkoxylated and in particular ethoxylated fatty alcohols, alkoxylated and in particular ethoxylated fatty esters, glycerolated esters or ethers (such as, for example, polyglycerol-4 isostearate), salts of fatty acids, such as aluminum stearate, sugar-derived surfactants, such as methyl glucose isostearate, polymeric surfactants derived from polyolefins and silicone emulsifiers and their mixtures.

For the polymeric surfactants derived from polyolefins, the non-polar part is selected from polyolefins, such as polymers and/or copolymers of ethylene, propylene, 1-butene, isobutene, 1-pentene, 2-methyl-1-butene, 3-methyl-1-butene, 1-hexene, 1-heptene, 1-octene, 1-decene, 1-undecene, 1-dodecene, 1-tridecene, 1-tetradecene, 1-pentadecene, 1-hexadecene, 1-heptedecene and 1-octadecene. The polymer chains are or are not hydrogenated. They are composed of at least 40 carbons and preferably of 60 to 700 carbons.

The polar part of these polymeric surfactants can be anionic, cationic, nonionic, zwitterionic or amphoteric. It is, for example, composed of acrylic derivatives, of polyalkylene glycols or polyalkyleneimine. The polymeric surfactants comprising a carboxylic acid polar part result, for example, from the reaction of a polyolefin with carboxylic acids, such as maleic acid, maleic anhydride, fumaric acid, itaconic acid, citraconic acid, mesaconic acid, aconitic acid, and the like. Preferably, their polar part is composed of succinic acid or anhydride, their ester or amide derivatives, the corresponding salts of alkali metal, alkaline earth metal or organic ions, or alternatively of polyoxyethylene.

The polymeric surfactants derived from polyolefins are also selected from the polyolefin derivatives of succinic acid described in U.S. Pat. Nos. 4,234,435, 4,708,753, 5,129,972, 4,931,110, 4,919,179 and GB-2,156,799. The polyolefin part can be composed of hydrogenated or non-hydrogenated polyisobutylene. The succinic anhydride or acid can be modified by alcohols, amines, alkanolamines or polyols, or can also occur in the form of salts of alkali metal or alkaline earth metal ions, or also of organic ions, such as the diethanolammonium or triethanolammonium ions. Mention may in particular be made of the polyisobutylenes comprising a modified succinic ending, such as the products marketed under the trademarks L2724, L2721, L2722, OS156565 and Lubrizol 5603 by Lubrizol or also Chemcinnate 2000, supplied by Chemron. Another example of a polymeric surfactant which can be used in the invention is the product of the reaction of maleic anhydride with polyisobutylene, such as Glissopal SA, marketed by BASF.

The silicone emulsifiers can be selected, for example, from the group consisting of dimethicone copolyols, alkyl dimethicone copolyols which can comprise heteroatoms, such as fluorine, and the mixtures comprising them, for example the polyglyceryl-4 isostearate/cetyl dimethicone copolyol/hexyl laurate mixture marketed under the trademark Abil WE 09 by Goldschmidt, the cetyl dimethicone copolyol marketed under the trademark Abil EM 90 by Goldschmidt and the cyclomethicone/dimethicone copolyol mixture marketed under the trademark Q2-3225C by Dow Corning. The level of emulsifier in the primary emulsion generally ranges from 0.1% to 10% by weight of active material and preferably from 0.5% to 5% by weight, with respect to the total weight of the primary emulsion.

The compositions of the invention can comprise all the additives commonly used in cosmetics and will find applications in the care field, makeup field and field of anti-sun products.

The aqueous compositions in accordance with the present invention can additionally comprise conventional cosmetic adjuvants selected in particular from organic solvents, ionic or nonionic and hydrophilic or lipophilic thickeners, softening agents, moisturizing agents, opacifiers, stabilizing agents, emollients, silicones, anti-foaming agents, fragrances, preservatives, anionic, cationic, nonionic, zwitterionic or amphoteric surfactants, active principles, fillers, polymers, propellants, basifying or acidifying agents or any other ingredient generally used in the cosmetic and/or dermatological field.

Mention may be made, as hydrophilic thickeners, of carboxyvinyl polymers, such as the Carbopols (carbomers) and the Pemulens (acrylate/C₁₀-C₃₀ alkyl acrylate copolymer); the terpolymer of methacrylic acid, methyl acrylate and dimethyl(meta-isopropenyl)benzyl isocyanate of ethoxylated alcohol (INCI name: Polyacrylate-3), such as the product marketed by Amerchol under the trademark Viscophobe DB 1000; polyacrylamides, such as, for example, the crosslinked copolymers marketed under the trademarks Sepigel 305 (CTFA name: polyacrylamide/C13-14 isoparaffin/Laureth 7) or Simulgel 600 (CTFA name: acrylamide/sodium acryloyldimethyltaurate copolymer/isohexadecane/polysorbate 80) by Seppic; optionally crosslinked and/or neutralized polymers and copolymers of 2-acrylamido-2-methylpropanesulfonic acid, such as the poly(2-acrylamido-2-methylpropanesulfonic acid) marketed by Clariant under the trademark “Hostacerin AMPS” (CTFA name: ammonium polyacryldimethyltauramide); cellulose derivatives, such as hydroxyethylcellulose; polysaccharides and in particular gums, such as xanthan gum; and their mixtures.

Mention may be made, as lipophilic thickeners, of synthetic polymers, such as the poly(C₁₀-30 alkyl acrylate) marketed under the trademark “Doresco IPA 13-1” by Landec, or also of modified clays, such as hectorite and its derivatives, for example the products marketed under the Bentone names.

The organic solvents can be selected from the group consisting of hydrophilic organic solvents, lipophilic organic solvents, amphiphilic solvents and their mixtures.

Mention may be made, among hydrophilic organic solvents, for example, of linear or branched monohydric alcohols having from 1 to 8 carbon atoms, such as ethanol, propanol, butanol, isopropanol or isobutanol; polyethylene glycols having from 6 to 80 ethylene oxides; polyols, such as propylene glycol, isoprene glycol, butylene glycol, glycerol or sorbitol; mono- or dialkyl isosorbides, the alkyl groups of which have from 1 to 5 carbon atoms, such as dimethyl isosorbide; or glycol ethers, such as diethylene glycol monomethyl or monoethyl ether and propylene glycol ethers, such as dipropylene glycol methyl ether.

Mention may be made, as ampphihilic organic solvents, of polypropylene glycol (PPG) derivatives, such as esters of polypropylene glycol and of fatty acid or ethers of PPG and of fatty alcohol, such as PPG-23 oleyl ether and PPG-36 oleate.

Mention may be made, as lipophilic organic solvents, for example, of fatty esters, such as diisopropyl adipate, dioctyl adipate or alkyl benzoates.

Mention may be made, as preservatives, of esters of para-hydroxybenzoic acid, also known as Parabens® (in particular methylparaben, ethylparaben or propylparaben), phenoxyethanol, releasers of formaldehyde, such as, for example, imidazolidinyl urea or diazolidinyl urea, chlorhexidine digluconate, sodium benzoate, caprylyl glycol, iodopropynyl butylcarbamate, pentylene glycol, alkyltrimethylammonium bromide, such as myristyltrimethylammonium bromide (CTFA name: myrtrimonium bromide), dodecyltrimethylammonium bromide, hexadecyltrimethylammonium bromide and their mixtures, such as the mixture marketed under the trademark Cetrimide® by FEF Chemicals. The preservative can be present in the composition according to the invention in a content ranging from 0.001% to 10% by weight, with respect to the total weight of the composition, in particular ranging from 0.1% to 5% by weight and especially ranging from 0.2% to 3% by weight.

Mention may be made, as fillers which can be used in the composition of the invention, for example, of pigments; silica powder; talc; polyamide particles and in particular those marketed under the trademark Orgasol by Atochem; polyethylene powders; powders formed of natural organic materials, such as starch powders, in particular powders formed of crosslinked or non-crosslinked maize, wheat or rice starches, such as powders formed of starch crosslinked by octenylsuccinic anhydride, marketed under the trademark Dry-Flo by National Starch; microspheres based on acrylic copolymers, such as those made of ethylene glycol dimethacrylate/lauryl methacrylate copolymer marketed by Dow Corning under the trademark of Polytrap; poly(methyl methacrylate) powders, such as those marketed under the trademark Micropearl M 100 by Matsumoto; expanded powders, such as hollow microspheres and in particular the microspheres marketed under the trademark Expancel by Kemanord Plast or under the trademark Micropearl F 80 ED by Matsumoto; silicone resin microbeads, such as those marketed under the trademark Tospearl by Toshiba Silicone; polyurethane powders, such as the powder formed of hexamethylene diisocyanate/trimethylol hexyllactone copolymer marketed under the trademark Plastic Powder D-400 by Toshiba Pigment (CTFA name: HDI/Trimethylol Hexyllactone Crosspolymer); and their mixtures. When they are present, these fillers can be in amounts ranging from 0.001% to 20% by weight, preferably from 0.1% to 10% by weight and better still from 1% to 5% by weight, with respect to the total weight of the composition.

Mention may be made, among active principles, of:

vitamins (A, C, E, K, PP, and the like) and their derivatives or precursors, alone or as mixtures;

agents for combating pollution and/or agents for combating free radicals;

depigmenting agents and/or propigmenting agents;

anti-glycation agents;

soothing agents;

NO-synthase inhibitors;

agents which stimulate the synthesis of dermal or epidermal macromolecules and/or which prevent their decomposition;

agents which stimulate the proliferation of fibroblasts;

agents which stimulate the proliferation of keratinocytes;

muscle-relaxing agents;

tightening agents;

matifying agents;

keratolytic agents;

desquamating agents;
moisturizing agents;

anti-inflammatory agents;

agents which act on the energy metabolism of the cells;

insect repellents;

substance P or substance CRGP antagonists;

agents for combating hair loss and/or for the regrowth of the hair;

anti-wrinkle agents.

Of course, one skilled in the art will take care to choose the additional optional compound or compounds mentioned above and/or their amounts so that the advantageous properties intrinsically attached to the compositions in accordance with the invention are not, or not substantially, detrimentally affected by the envisaged addition or additions.

The present invention also features the use of the compositions according to the invention as defined above in the manufacture of products for the cosmetic treatment of the skin, lips, nails, hair, eyelashes, eyebrows and/or scalp, in particular care products, sun protection products and makeup products.

The compositions according to the invention can constitute a product for caring for the skin, in particular for the face, neck, outline of the eye or body; or alternatively a makeup product, such as a product for the complexion (in particular foundation), concealer or sun protection product; or indeed a product for cleaning the skin. Preferably, the composition according to the invention will be a sun protection product.

The cosmetic compositions according to the invention can, for example, be used as care product and/or sun protection product for the face and/or the body with a liquid to semi-liquid consistency, such as lotions, milks, more or less smooth creams, gels or cream gels. They can also be packaged in an aerosol and be provided in the foam or spray form.

The compositions according to the invention in the form of vaporizable fluid lotions in accordance with the invention are applied to the skin or hair in the form of fine particles using pressurization devices. The devices in accordance with the invention are well known to a person skilled in the art and comprise non-aerosol pumps or “atomizers”, aerosol containers comprising a propellant and aerosol pumps using compressed air as propellant. The latter are described in U.S. Pat. Nos. 4,077,441 and 4,850,517 (forming an integral part of the content of the description).

The compositions packaged in aerosol in accordance with the invention generally comprise conventional propellant, such as, for example, hydrofluorinated compounds, dichlorodifluoromethane, difluoroethane, dimethyl ether, isobutane, n-butane, propane or trichlorofluoromethane. They are preferably present in amounts ranging from 15% to 50% by weight, with respect to the total weight of the composition.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

| | | Formula | | |
|---|---|---|---|---|
| | | (outside | | |
| | | the | Example | Example |
| Phase | Constituents | invention) | 1 | 2 |
| A | Distilled water | 15.375% | 15.375% | 15.375% |
| | Preservatives | 0.8% | 0.8% | 0.8% |
| | Glycerol | 5.00% | 5.00% | 5.00% |
| | EDTA | 0.20% | 0.20% | 0.20% |
| B | Sucrose distearate | 2.00% | 2.00% | 2.00% |
| | Sorbitan stearate | 1.00% | 1.00% | 1.00% |
| | 20 EO | | | |
| | N-Stearoyl-L- | 0.75% | 0.75% | 0.75% |
| | glutamic acid, | | | |
| | monosodium salt | | | |
| | Preservative | 0.15% | 0.15% | 0.15% |
| | Cyclohexasiloxane | 1.72% | 1.72% | 1.72% |
| | Diisopropyl sebacate | 2.71% | 2.71% | 2.71% |
| | Isononyl | 5.57% | 5.57% | 5.57% |
| | isononanoate | | | |
| | Octocrylene | 7.00% | 7.00% | 7.00% |
| | Butylmethoxydiben- | 3.00% | 3.00% | 3.00% |
| | zoylmethane | | | |
| | Ethylhexyl salicylate | 5.00% | 5.00% | 5.00% |
| C | Distilled water | 15.00% | 15.00% | 15.00% |
| | Carbomer 980 | 0.30% | 0.30% | 0.30% |
| | Triethanolamine | 0.30% | 0.30% | 0.30% |
| D | Distilled water | 6.00% | 6.00% | 6.00% |
| | Xanthan gum | 1.60% | 1.60% | 1.60% |
| E | PEG 12 dimethicone | 0.50% | 0.50% | 0.50% |
| F | Aluminum starch | 3.00% | 3.00% | 3.00% |
| | octenylsuccinate | | | |
| G | Takemoto NLK 506 | 0 | 3.00% | 5.00% |
| H | Distilled water | q.s. for | q.s. for | q.s. for |
| | | 100 | 100 | 100 |
| SPF | | 14.4 +/− | 24.3 +/− | 33.8 +/− |
| in | | 0.9 | 3.1 | 3.7 |
| vitro | | | | |

The sun protection factor (SPF) is determined according to the “in vitro” method described by B. L. Diffey in J. Soc. Cosmet. Chem. 40,127-133 (1989). The measurements were carried out using a UV-1000S spectrophotometer from Labsphere. Each composition is applied to a quartz slide in the form of a homogeneous and uniform deposit in a proportion of 1.33 mg/cm².

It is found that the SPF is significantly improved when hollow hemispherical particles are added to the reference composition. The in vitro SPF values are improved by 68% to 134% respectively with 3% and 5% of Takemoto NLK 506 particles. Furthermore, these particles make it possible to significantly reduce the shine, after application, of the screening formulations comprising them.

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. An increased SPF sunscreen composition comprising at least one aqueous phase, at least one organic UV screening agent and/or at least one inorganic UV screening agent, and an effective SPF-increasing amount of concave or annular particles of a silicone material, formulated into a physiologically accepetable medium therefor.

2. The sunscreen composition as defined by claim 1, wherein the particles of silicone material are in combination with at least one wetting agent.

3. The sunscreen composition as defined by claim 1, comprising concave particles of silicone material that have a mean diameter of less than or equal to 10 μm.

4. The sunscreen composition as defined by claim 3, wherein the concave particles of silicone material have a mean diameter ranging from 0.1 μm to 8 μm.

5. The sunscreen composition as defined by claim 3, wherein the concave particles of silicone material are in the form of portions of hollow spheres having a transverse cross-section with the shape of a horseshoe or arch.

6. The sunscreen composition as defined by claim 1, wherein the silicone material comprises a crosslinked polysiloxane with a three-dimensional structure comprising or composed of units of formula (I): SiO₂ and of formula (II):
R¹SiO_{1.5}
in which R¹ is an organic group having a carbon atom directly bonded to the silicon atom.

7. The sunscreen composition as defined by claim 6, wherein R¹ is a C₁-C₄ alkyl group or a phenyl group.

8. The sunscreen composition as defined by claim 7, wherein R¹ is a methyl group.

9. The sunscreen composition as defined by claim 6, wherein R¹ is selected from among epoxy, (meth)acryloyloxy, alkenyl, mercaptoalkyl, aminoalkyl, haloalkyl, glyceroxy, ureido or cyano groups.

10. The sunscreen composition as defined by claim 6, wherein the silicone material comprises the units (I) and (II) according to a unit (I)/unit (II) molar ratio ranging from 30/70 to 50/50.

11. The sunscreen composition as defined by claim 1, comprising concave particles made of silicone material prepared by a process which comprises:
(a) the introduction into an aqueous medium, in the presence of at least one hydrolysis catalyst and optionally of at least one surfactant, of a compound (III) of formula SiX₄ and of a compound (IV) of formula RSiY₃, where X and Y denote, independently of one another, a C₁-C₄ alkoxy group, an alkoxyethoxy group including a C₁-C₄ alkoxy group, a C₂-C₄ acyloxy group, an N,N-dialkylamino group including a C₁-C₄ alkyl group, a hydroxyl group, a halogen atom or a hydrogen atom and R is an organic group comprising a carbon atom bonded directly to the silicon atom; and
(b) the operation in which the mixture resulting from stage (a) is contacted with an aqueous solution including at least one polymerization catalyst and optionally at least one surfactant, at a temperature of from 30° to 85° C., for at least two hours.

12. The sunscreen composition as defined by claim 11, wherein stage (a), the molar ratio of the compound (III) to the compound (IV) ranges from 30/70 to 50/50.

13. The sunscreen composition as defined by claim 11, wherein the ratio by weight of the water to the total of the compounds (III) and (IV) ranges from 10/90 to 70/30 in stage (a).

14. The sunscreen composition as defined by claim 11, wherein R is a C₁-C₄ alkyl group or a phenyl group.

15. The sunscreen composition as defined by claim 14, wherein R is a methyl group.

16. The sunscreen composition as defined by claim 11, wherein R is selected from among epoxy, (meth)acryloyloxy, alkenyl, mercaptoalkyl, aminoalkyl, haloalkyl, glyceroxy, ureido or cyano groups.

17. The sunscreen composition as defined by claim 1, comprising concave particles formed of a small internal arc (11), of a large external arc (21) and of segments (31) which connect the ends of the respective arcs, the width (W1) from the two ends of the small internal arc (11) ranging from 0.01 to 8 μm on average, the width (W2) from the two ends of the large external arc (21) ranging from 0.05 to 10 μm on average, and the height (H) of the large external arc (21) ranging from 0.015 to 8 μm on average.

18. The sunscreen composition as defined by claim 1, comprising particles of annular shape that have a mean external diameter of 0.05 to 15 μm and a mean internal diameter of 0.01 to 10 μm, the difference from the mean external diameter and the mean internal diameter being from 0.04 to 5 μm.

19. The sunscreen composition as defined by claim 1, said particles exhibiting a polysiloxane network comprising siloxane units of formulae (1), (2), (3), (4), (5) and (6):
SiO_{4/2} (1)
Si(OH)_{3/2} (2)
R₁SiO_{3/2} (3)
R₂SiO_{3/2} (4)
R₃SiO_{3/2} (5)
R₄SiO_{3/2} (6)
in which R₁ and R₃ denote alkyl, cycloalkyl, aryl, alkylaryl or aralkyl groups and R₂ and R₄ each denote a hydrocarbon group selected from among the acryloyloxy, methacryloyloxy, vinyl or mercapto groups;
the siloxane units of formula (1)/siloxane units of formulae (2), (3), (4), (5) and (6) molar ratio being from 20/80 to 50/50;
the siloxane units of formulae (2), (3) and (4)/siloxane units of formulae (5) and (6) molar ratio being from 50/50 to 75/25; and
the siloxane units of formulae (3) and (5)/siloxane units of formulae (4) and (6) molar ratio being from 20/80 to 60/40.

20. The sunscreen composition as defined by claim 2, wherein the wetting agent is selected from among water-soluble silicones comprising at least one terminal or pendent monovalent polyoxyalkylene group.

21. The sunscreen composition as defined by claim 20, wherein the wetting agent is selected from among water-soluble silicones comprising at least one polyoxyalkylene group of the following general formula (a):
R²₃SiO(R²₂SiO)ₚ(R²PESiO)_{q}SiR²₃ (a)
in which:
the R² radicals, which are identical or different, denote a monovalent hydrocarbon radical selected from alkyl, aryl and aralkyl radicals having at most 10 carbon atoms; certain of the R² radicals can also additionally comprise an ethylcyclohexylene monoxide group of formula: 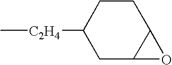
and are in low proportion in the polysiloxane chain;
p varies from 0 to 150;
q varies from 1 to 12;
the polyether group PE has the following formula (b):
—CₓH₂ₓ(OC₂H₄)_{y}(OC₃H₆)_{z}OR³ (b)
in which:
x varies from 1 to 8;
y is greater than 0;
z is greater than or equal to 0; the values of y and z are such that the total molecular weight of the polyoxyalkylene portion of the polyether group PE varies from 200 to 10,000;
R³ is hydrogen, a C₁-C₈ alkyl group or a C₂-C₈ acyl group.

22. The sunscreen composition as defined by claim 21, wherein the R² radicals are selected from among lower C₁-C₆ alkyls, phenyl and benzyl.

23. The sunscreen composition as defined by claim 22, wherein the R² radicals are selected from among lower C₁-C₄ alkyls.

24. The sunscreen composition as defined by claim 21, wherein the R³ radicals are selected from among lower C₁-C₄ alkyls.

25. The sunscreen composition as defined by claim 21, wherein the water-soluble silicones of formula (a) are selected from among those of following formula (a′):
Me₃SiO(MeSiO)ₚ(MePESiO)_{q}SiMe₃ (a′)
where Me is methyl; PE is the group of formula (b′):
—(CH₂)₃(OC₂H₄)_{y}(OC₃H₆)_{z}OR³ (b′)
R³ is hydrogen or a C₁-C₄ alkyl group.

26. The sunscreen composition as defined by claim 20, wherein the water-soluble silicone is selected from among the branched silicones of following formula (c):
(MeSiO)_{q-2}[SiOMe₂)_{p/q}OPE]_{q} (c)
wherein:
p varies from 0 to 150;
q varies from 1 to 12;
Me is methyl;
PE is the group of following formula (d):
—(OC₂H₄)_{y}(OC₃H₆)_{z}R³ (d)
wherein:
y is greater than 0;
z is greater than or equal to 0; the values of y and z being such that the total molecular weight of the polyoxyalkylene moiety of the polyether group PE varies from 200 to 10,000; and
R³ is a C₁-C₄ alkyl group.

27. The sunscreen composition as defined by claim 1, comprising at least one organic UV screening agent selected from the group consisting of anthranilates; dibenzoylmethane derivatives; cinnamic derivatives; salicylic derivatives; camphor derivatives; benzophenone derivatives; β,β-diphenylacrylate derivatives; triazine derivatives; benzotriazole derivatives; benzalmalonate derivatives; benzimidazole derivatives; imidazolines; bis-benzoazolyl derivatives; p-aminobenzoic acid (PABA) derivatives; methylenebis(hydroxyphenylbenzotriazole) derivatives; benzoxazole derivatives; screening polymers and screening silicones; dimers derived from α-alkylstyrene; 4,4-diarylbutadienes; and mixtures thereof.

28. The sunscreen composition as defined by claim 27, wherein the at least one organic UV screening agent is selected from the group consisting of:
Ethylhexyl Methoxycinnamate,
Homosalate,
Ethylhexyl Salicylate,
Octocrylene,
Phenylbenzimidazole Sulfonic Acid,
Benzophenone-3,
Benzophenone-4,
Benzophenone-5,
n-Hexyl 2-(4-diethylamino-2-hydroxybenzoyl)benzoate,
4-Methylbenzylidene Camphor,
Terephthalylidene Dicamphor Sulfonic Acid,
Disodium Phenyl Dibenzimidazole Tetrasulfonate,
Ethylhexyl triazone,
Bis-Ethylhexyloxyphenol Methoxyphenyl Triazine,
Diethylhexyl Butamido Triazone,
2,4,6-Tris(dineopentyl 4′-aminobenzalmalonate)-s-triazine,
2,4,6-Tris(diisobutyl 4′-aminobenzalmalonate)-s-triazine,
2,4,6-Tris(biphenyl-4-yl)-1,3,5-triazine,
2,4,6-Tris(terphenyl)-1,3,5-triazine,
Methylene Bis-Benzotriazolyl Tetramethylbutylphenol,
Drometrizole Trisiloxane,
Polysilicone-15,
Dineopentyl 4′-methoxybenzalmalonate,
1,1-Dicarboxy(2,2′-dimethylpropyl)-4,4-diphenylbutadiene,
2,4-Bis[5-1 (dimethylpropyl)benzoxazol-2-yl-(4-phenyl)imino]-6-(2-ethylhexyl)imino-1,3,5-triazine,
and their mixtures.

29. The sunscreen composition as defined by claim 1, comprising at last one inorganic UV screening agent selected from among metal oxide pigments which may or may not be coated.

30. The sunscreen composition as defined by claim 29, wherein said pigments are selected from among titanium oxide, zinc oxide, iron oxide, zirconium oxide, cerium oxide and their mixtures, which may or may not be coated.

31. The sunscreen composition as defined by claim 30, wherein the pigments are based on titanium oxide, which may or may not be coated.

32. The sunscreen composition as defined by claim 31, wherein the titanium oxide is in the rutile, anatase or amorphous form.

33. The sunscreen composition as defined by claim 29, wherein the pigments have a mean elementary particle size of greater than 5 nm and less than 100 nm.

34. The sunscreen composition as defined by claim 1, in the form of an oil-in-water emulsion, a water-in-oil emulsion, a water-in-oil-in-water triple emulsion or an oil-in-water-in-oil triple emulsion.

35. The sunscreen composition as defined by claim 34, in the form of an oil-in-water emulsion or of a water-in-oil-in-water triple emulsion.

36. The sunscreen composition as defined by claim 1, wherein the concave or annular particles are present in a content ranging from 0.1% to 15% by weight, with respect to the total weight of the composition.

37. The sunscreen composition as defined by claim 1, wherein the UV screening agent or agents are present in proportions ranging from 0.01 to 20% by weight, with respect to the total weight of the composition.

38. The sunscreen composition as defined by claim 1, wherein the proportion by weight of the aqueous phase varies from 25% to 95% by weight with respect to the total weight of the composition.

39. The sunscreen composition as defined by claim 2, wherein the wetting agent is selected from among water-soluble silicones comprising at least one terminal or pendent monovalent polyoxyalkylene group.

40. The sunscreen composition as defined by claim 1, further comprising at least one cosmetic adjuvant selected from the group consisting of organic solvents, ionic or nonionic and hydrophilic or lipophilic thickeners, softening agents, moisturizing agents, opacifiers, stabilizing agents, emollients, silicones, anti-foaming agents, fragrances, preservatives, anionic, cationic, nonionic, zwitterionic or amphoteric surfactants, active principles, fillers, polymers, propellants, basifying or acidifying agents and mixtures thereof.

41. The sunscreen composition as defined by claim 1, in the form of a white or colored cream, of a milk, of a lotion, of a cream gel or in a solid form.

42. The sunscreen composition as defined by claim 1, packaged in an aerosol and can form a foam or a spray.

43. The sunscreen composition as defined by claim 1, in the form of products for the cosmetic treatment of the skin, lips, nails, hair, eyelashes, eyebrows and/or scalp.

44. The sunscreen composition as defined by claim 1, in the form of a care product and/or sun protection product for the face and/or body.

45. The sunscreen composition as defined by claim 1, comprising a makeup product.

46. The sunscreen composition as defined by claim 1, said particles comprising portions of hollow spheres.
